(19) United States
(12) Patent Application Publication
(10) Pub. No.: US 2024/0115846 A1
(43) Pub. Date: Apr. 11, 2024

(54) HEMOSTASIS VALVE, INTRODUCER AND RETRIEVAL DEVICE

(71) Applicant: Silara Medtech Inc., Santa Rosa, CA (US)

(72) Inventors: Mark GEUSEN, Santa Rosa, CA (US); Luis A. HERNANDEZ, Santa Rosa, CA (US); Carl POPPE, Santa Rosa, CA (US)

(21) Appl. No.: 18/261,894

(22) PCT Filed: Jan. 12, 2022
(86) PCT No.: PCT/US2022/012063

§ 371 Date: Jul. 18, 2023

Related U.S. Application Data
(60) Provisional application No. 63/139,124, filed on Jan. 19, 2021.

(51) Int. Cl. A61M 39/06; A61F 2/24

(52) U.S. Cl. CPC A61M 39/0613; A61F 2/2466; A61M 2039/062; A61M 2039/0673

(57) ABSTRACT

The present disclosure provides at least one hemostasis valve, an introducer, and a retrieval device. The hemostasis valve may include a sealing member, a pressing assembly and a rotary motion assembly. The sealing member may be configured to define a sealing cavity which is transversely contractible. The pressing assembly is disposed at an outer periphery of the sealing member. The pressing assembly is configured for linear motion in a transverse direction of the sealing cavity to change a dimension of the sealing cavity. The rotary motion assembly is coupled to the pressing assembly, and the rotary motion assembly may be configured to convert rotary motion into the linear motion of the pressing assembly through the coupling. The hemostasis valve of the present disclosure can perform active hemostasis sealing for one or more medical devices and is convenient to use.

### CROSS-REFERENCE OF RELATED APPLICATION

The present disclosure claims priority of U.S. Provisional Application No. 63/139,124 filed on Jan. 19, 2021, which is entirely incorporated herein by reference.

### TECHNICAL FIELD

The present disclosure relates to a technical field of medical facilities, and particularly to at least one hemostasis valve, an introducer and a retrieval device.

### BACKGROUND ART

The interventional catheter technology applied to a cardiovascular system often needs to use an introducer device to form an access port from a skin to a vein or an artery, and a catheter of an independent delivery system is introduced into a vasculature through the introducer device. The catheter of the delivery system may be employed to load a prosthetic heart valve, a crimped stent, an inflatable balloon, and other medical devices for deployment into the vasculature of a patient. The introducer device is usually provided with a hemostasis sealing member through which the catheter enters the vasculature. In addition to the introducer, other catheter-like human intervention devices also usually need to be provided with hemostasis sealing members.

### SUMMARY OF THE DISCLOSURE

An objective of the present disclosure is to provide at least one hemostasis valve, an introducer comprising the hemostasis valve, and a retrieval device comprising the hemostasis valve.

In order to achieve the above objective, the present disclosure proposes a hemostasis valve. In some embodiments, the hemostasis valve may comprise a sealing member, a pressing assembly and a rotary motion assembly. The sealing member may be configured to define a sealing cavity which is transversely contractible. The pressing assembly is disposed at an outer periphery of the sealing member. The pressing assembly is configured for linear motion in a transverse direction of the sealing cavity to change a dimension of the sealing cavity. The rotary motion assembly is coupled to the pressing assembly. The rotary motion assembly may be configured to convert rotary motion into the linear motion of the pressing assembly through the coupling.

The present disclosure further proposes an introducer. In some embodiments, the introducer may comprises a housing, a sheath tube, the aforementioned hemostasis valve and a driving knob. The driving knob may be configured to operate the hemostasis valve. The hemostasis valve is disposed inside the housing. The sheath tube is connected to the sealing member of the hemostasis valve, and the sheath tube is arranged coaxially with and communicated with the sealing cavity of the sealing member. The driving knob is disposed outside the housing and attached to the rotary motion assembly of the hemostasis valve.

The present disclosure further proposes a retrieval device. According to some embodiments, the retrieval device may be configured to retrieve an implant in a human body. In some embodiments, the retrieval device may comprise a retrieval net, an inner tube, an outer tube, a handle, a slider, a sheath tube, and the aforementioned hemostasis valve. The retrieval net has a opened state and a closed state, and is connected to the inner tube. The outer tube may sleeve the inner tube, the outer tube and the inner tube are in sealing fit and movable axially relative to each other, and the retrieval net can be moved into or out of the outer tube by driving the outer tube and the inner tube to move axially relative to each other. The handle sleeves the outer tube. The slider is disposed outside the handle and in sliding fit with the handle. One of the outer tube and the inner tube is fixedly connected to the handle and the other is fixedly connected to the slider. The outer tube and the inner tube are driven to move axially relative to each other by sliding the slider. The sheath tube runs through the inner tube and in sealing fit with the inner tube. The sealing member of the hemostasis valve is connected to the sheath tube, and the sealing cavity of the sealing member is communicated with the sheath tube.

The characteristics and advantages of the hemostasis valve of the present disclosure include: being provided with a rotary motion assembly and a linear motion pressing assembly, the hemostasis valve of the present disclosure converts rotary motion into linear motion, so that active hemostasis sealing can be provided for one or more medical devices just by a single operator's operation. The operation is convenient and the sealing effect is significant.

### BRIEF DESCRIPTION OF THE DRAWINGS

The following drawings are only intended to illustrate and explain the present disclosure schematically, rather than limiting the scope of the present disclosure. In the drawings:

FIG. 1 illustrates a schematic view of a structure of an introducer in an embodiment of the present disclosure;

FIG. 2 illustrates a schematic view of a partial structural of an introducer with a hemostasis valve in an embodiment of the present disclosure;

FIG. 3 illustrates an exploded view of a partial structure of the introducer in FIG. 1;

FIG. 4 illustrates a cross-sectional view of a partial structure of the introducer in FIG. 2;

FIGS. 5 and 6 illustrate schematic views of a structure of one linear motion member of a hemostasis valve in an embodiment of the present disclosure;

FIGS. 7 to 9 illustrate schematic views of a structure of another linear motion member of a hemostasis valve in an embodiment of the present disclosure;

FIGS. 10 and 11 illustrate schematic views of a structure of a secondary gear in FIG. 2;

FIGS. 12 and 13 illustrate schematic views of a structure of a primary gear in FIG. 2;

FIGS. 14 to 17 illustrate schematic views of different states of motions of a pressing assembly when a manual driving knob is at different angles;

FIGS. 18 to 20 illustrate schematic views of a sealing member in FIG. 2;

FIG. 21 illustrates a schematic view of a first case in FIG. 2;

FIGS. 22 to 24 illustrate schematic views of an internal structure of a sealing assembly with a hemostasis valve in another embodiment of the present disclosure, wherein FIG. 24 illustrates a partial enlarged view of portion A in FIG. 23;

FIGS. 25 and 26 illustrate schematic views of a linear motion member in FIG. 22;

FIGS. 27 and 28 illustrate schematic views of another linear motion member in FIG. 22;

FIGS. 29 to 31 illustrate schematic views of a retrieval device in an embodiment of the present disclosure when a retrieval net is in a closed state, wherein FIG. 31 illustrates a partial enlarged view of portion B in FIG. 30;

FIGS. 32 and 33 illustrate schematic views of a structure of a retrieval device in an embodiment of the present disclosure when a retrieval net is in a opened state;

FIGS. 34 and 35 illustrate schematic views of a connection between a retrieval net and an inner tube in FIG. 32;

FIGS. 36 and 37 illustrate schematic views of a connection between an outer tube and a sliding member in FIG. 32, wherein FIG. 37 illustrates a cross-sectional view taken along line C-C in FIG. 36;

FIGS. 38 and 39 illustrate schematic views of a handle in FIG. 32.

### DETAILED DESCRIPTION OF THE PREFERRED EMBODIMENTS

In order that the technical features, objectives and effects of the present disclosure can be understood more clearly, the specific embodiments of the present disclosure will be described with reference to the drawings. Wherein, the terms ‘first’, ‘second’, etc. are only used for descriptive purposes, and cannot be understood as indicating or implying relative importance or implicitly indicating the number of the described technical features. Therefore, a feature limited by ‘first’, ‘second’, etc. can explicitly or implicitly include one or more of such features. In the description of the present disclosure, unless otherwise specified, the term ‘a plurality of’ means two or more; the term ‘proximal end’ means a position on a device closest to an operator (e.g., a doctor) who uses the device, whereas the term ‘distal end’ means a position on the device farthest from the operator.

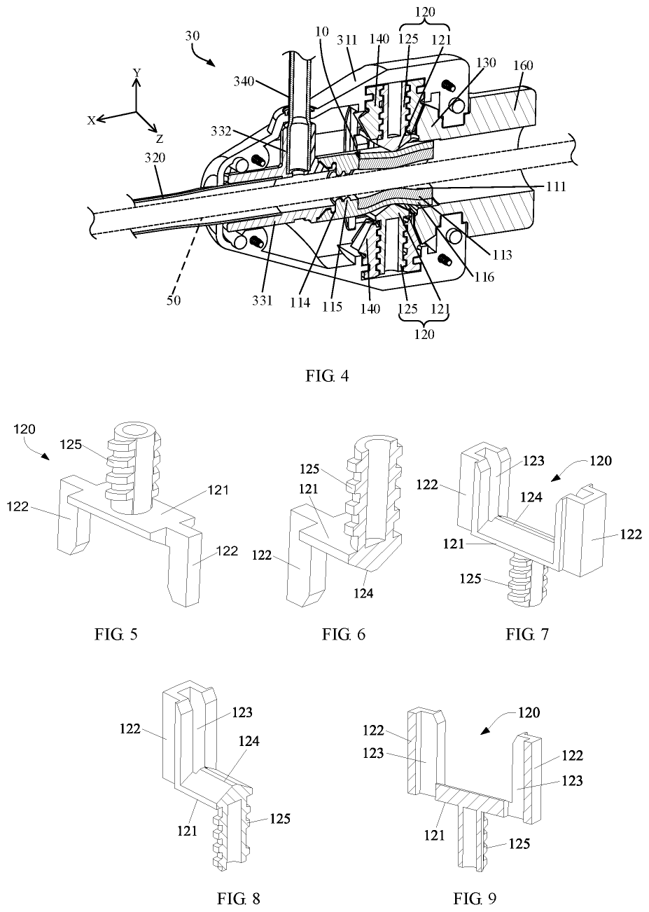

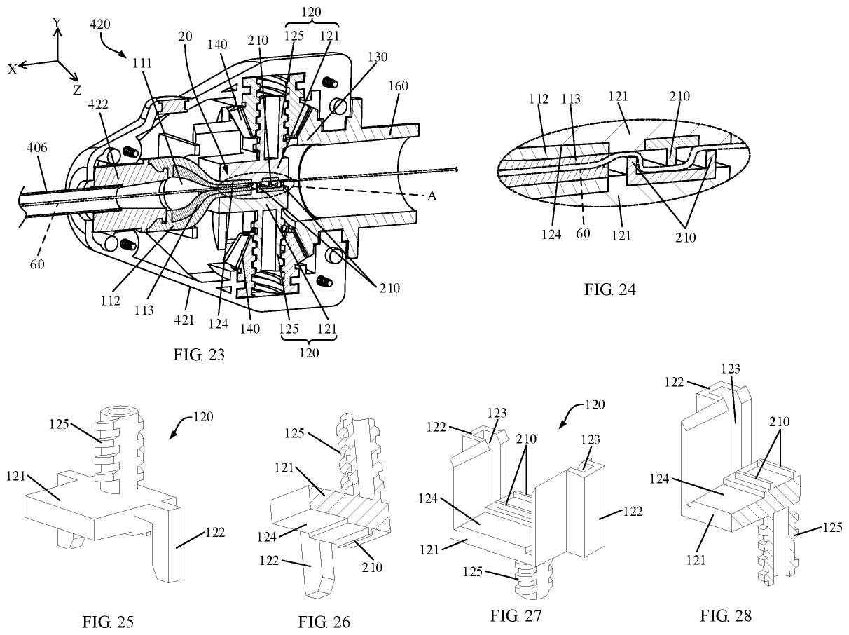

Referring to FIGS. 1 to 4, it is one aspect of the present disclosure to provide a hemostasis valve 10, comprising a sealing member 110, a pressing assembly and a rotary motion assembly. A sealing cavity 111, which can be transversely contracted, is provided in the sealing member 110 in a longitudinal direction. Referring to FIGS. 3 and 4, an X-axis direction, a Y-axis direction, and a Z-axis direction are three directions perpendicular to each other, wherein the X-axis direction is the longitudinal direction of the sealing cavity 111. The sealing cavity 111 allows one or more medical devices, such as a catheter 50 (as illustrated in FIG. 4) or a positioning guide wire 60 (as illustrated in FIG. 23), to pass through along the longitudinal direction. The sealing member 110 is an elastic sealing member, and for example, a material of the sealing member 110 is an elastic polymer. The sealing cavity 111 seals each medical device by a transverse contraction, so as to prevent a blood loss caused by a gap between the sealing cavity 111 and each medical device.

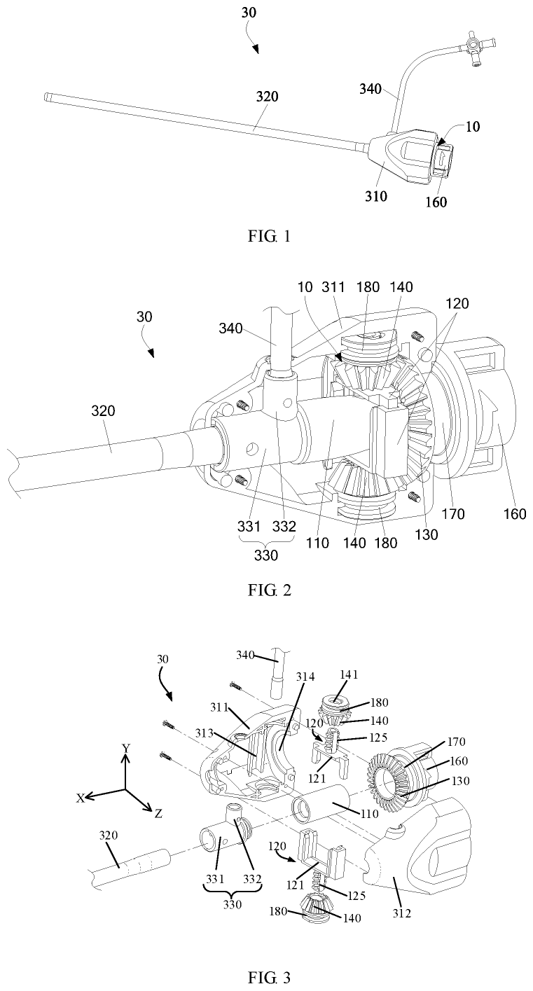

The pressing assembly is disposed at an outer periphery of the sealing member 110 and configured for linear motion in a transverse direction of the sealing cavity 111 to change a dimension of the sealing cavity 111. As exemplarily illustrated in FIG. 2, the pressing assembly comprises two pressing members 120 disposed on two opposite sides of the sealing member 110 and configured to clamp or loosen the sealing member 110 by moving toward or away from each other in the transverse direction of the sealing cavity 111 to change the dimension of the sealing cavity 111. The rotary motion assembly is coupled to the pressing assembly (i.e., the two pressing members 120 in FIG. 2) and configured to convert rotary motion into the linear motion of the pressing assembly through the coupling. The rotary motion assembly drives the pressing assembly for the linear motion toward a center of the sealing cavity 111, so that the pressing assembly compresses the sealing member 110 to drive the sealing cavity 111 to contract transversely, thereby realizing active sealing of one or more medical devices.

In the hemostasis valve 10 of the present disclosure, the pressing assembly is adopted to compress the sealing member 110 to drive the sealing cavity 111 to contract transversely. An inner side of the sealing cavity 111 of the compressed sealing member 110 is deformed, which can eliminate a gap between the sealing cavity 111 and one or more medical devices. The dimension of the sealing cavity 111 can be freely and flexibly adjusted by adjusting a movement amount of the pressing assembly. When one or more medical devices pass through the sealing cavity 111, the sealing member 110 can provide reliable sealing for all medical devices. The hemostasis valve of the present disclosure can actively seal a plurality of medical devices, and can achieve better sealing effect and convenient use.

In addition, since the dimension of the sealing cavity 111 can be flexibly adjusted, the hemostasis valve has the valve characteristics of being openable and closeable, and adjustable in an opening degree. When the pressing assembly does not compress the sealing member 110, the sealing cavity 111 has an original aperture, i.e., a maximum aperture. At this time, the sealing cavity 111 is in a fully open state, that is, the hemostasis valve is in a fully open state, and the operator can move one or more medical devices passing through the sealing cavity 111. When the pressing assembly compresses the sealing member 110 until the sealing cavity 111 is tightly attached to each medical device, the sealing cavity 111 is in a sealed state, that is, the hemostasis valve is in a sealed state. When the pressing assembly compresses the sealing member 110 until the aperture of the sealing cavity 111 is zero (at this time, no medical device passes through the sealing cavity 111), the sealing cavity 111 is in a completely closed state, that is, the hemostasis valve is in a completely closed state, so as to avoid a blood loss from the sealing cavity 111 caused by waiting for a medical device to pass through the sealing cavity 111 during a surgical procedure.

In some embodiments, the cross section of the sealing cavity 111 may have a circular shape, and the sealing member 110 may have a cylindrical shape, so as to improve the sealing effect on the medical device. As exemplarily illustrated in FIGS. 3 and 4, the X-axis direction is the axial direction of the sealing member 110 and the sealing cavity 111. However, the present disclosure is not limited thereto. In other embodiments, the cross section of the sealing cavity 111 may also have an elliptic shape or any other shape with a smooth inner wall surface, and the sealing member 110 may also have an elliptic cylinder shape.

In some embodiments, the pressing assembly may comprise at least one linear motion member with a part coupled to the rotary motion assembly, and another part providing an abutting portion for the sealing member 110 for changing the dimension of the sealing cavity 111. Each linear motion member is in contact with the sealing member 110 through its own abutting portion, and the rotary motion assembly converts the rotary motion into linear motion of each linear motion member through the coupling. Each linear motion member changes the dimension of the sealing cavity 111 by a linear motion in the transverse direction of the sealing cavity 111.

As exemplarily illustrated in FIGS. 2 to 4, the pressing member 120 is a specific implementation of the linear motion member. The pressing member 120 may comprise a pressing portion 121, which serves as said another part of the linear motion member to provide an abutting portion 124 (refer to FIGS. 6 to 7). When the rotary motion assembly drives the pressing member 120 for linear motion toward the center of the sealing cavity 111, the abutting portion 124 compresses the sealing member 110, so that the sealing cavity 111 contracts transversely. When the rotary motion assembly drives the pressing member 120 for linear motion away from the center of the sealing cavity 111, the abutting portion 124 withdraws the compression on the sealing member 110, and the sealing cavity 111 recovers from the contracted state to the original state by its elasticity.

In some embodiments, the pressing assembly may be configured to comprise a first linear motion member and a second linear motion member which are oppositely disposed. The first linear motion member may be configured to comprise a first pressing portion having a first abutting portion. The second linear motion member may be configured to comprise a second pressing portion having a second abutting portion.

As exemplarily illustrated in FIGS. 2 to 4, the two pressing members 120 may be disposed on two opposite sides of the sealing member 110 as the first linear motion member and the second linear motion member, respectively. The pressing portions 121 of the two pressing members 120 may serve as the first pressing portion and the second pressing portion, respectively, to jointly clamp and compress the sealing member 110. The abutting portions 124 of the two pressing portions 121 may serve as the first abutting portion and the second abutting portion, respectively, to contact with an outer side of the sealing member 110. Being driven by the rotary motion assembly, the two pressing members 120 move toward (moving in opposite directions) or away from (moving in reverse directions) each other to clamp or loosen the sealing member 110 quickly.

Further, the first pressing portion may be provided with a first guide rod, and the second pressing portion may be provided with a second guide rod. The first guide rod and the second guide rod may extend and guide in the transverse direction of the sealing cavity 111, and are in sliding fit to guide the linear motion of the linear motion member.

As exemplarily illustrated in FIGS. 2, 5 and 7, each pressing portion 121 may be provided with two guide rods 122, which are fixed at two opposite ends of the pressing portion 121, respectively. The two guide rods 122 may be oppositely disposed and parallel to each other, and may both be perpendicular to the pressing portion 121. The two pressing portions 121 may be respectively located on the two opposite sides of the sealing member 110 in a Y-axis direction and jointly clamp the sealing member 110, and the two guide rods 122 on each pressing portion 121 may be respectively located on the two opposite sides of the sealing member 110 in a Z-axis direction. Therefore, the two pressing members 120 clamp and surround the sealing member 110 from upper, lower, left and right directions, and the guide rods 122 of the two pressing members 120 are slidably fitted in the transverse direction of the sealing cavity 111 to guide the pressing members 120 for linear motion in the transverse direction of the sealing cavity 111, thereby ensuring a smooth movement of the pressing members 120.

In addition, the guide rods 122 of the two pressing members 120 can also limit the size of the sealing member 110 in the Z-axis direction, so as to prevent the sealing member 110 from being excessively extended in the Z-axis direction when being extruded, and enhance the sealing effect.

Specifically, the two guide rods 122 (hereinafter referred to as the first guide rods) of one of the two pressing members 120 (refer to FIGS. 7 to 9) may be respectively provided with a sliding groove 123, an extending direction of which is the transverse direction of the sealing cavity 111. Referring to FIG. 4, the two guide rods 122 (hereinafter referred to as the second guide rods) of the other one of the two pressing members 120 (refer to FIGS. 5 and 6) are respectively inserted into the two sliding grooves 123 of the two first guide rods and in sliding fit therewith, thereby realizing sliding fit between the first guide rods and the second guide rods. However, the present disclosure is not limited thereto. In other embodiments, any other existing sliding fit structure may also be applied.

In other embodiments, the pressing assembly may be further configured to comprise one linear motion member or more than two linear motion members.

For example, when the pressing assembly is configured to comprise one linear motion member, that is, when one pressing member 120 is provided, a supporting member may be disposed on a fixed case (e.g., a first housing 310 of the introducer 30) where the hemostasis valve 10 is mounted. The supporting member is fixedly connected to the fixed case, and fitted with the pressing portion 121 of the pressing member 120, so that the supporting member and the pressing portion 121 are cooperated to extrude the sealing member 110. The structure of the supporting member may be set with reference to that of the structure of the pressing portion 121.

When the pressing assembly is configured to comprise more than two linear motion members, these linear motion members are uniformly distributed along a circumferential direction of the sealing member 110, and jointly compress the sealing member 110 by motioning toward the center of the sealing cavity 111, so that the sealing cavity 111 is contracted transversely. By providing more than two linear motion members, it is beneficial to realize the uniform and rapid radial contraction of the sealing cavity 111, while leading to a complex structure of the hemostasis valve 10. Therefore, an embodiment in which two linear motion members are provided is the optimal embodiment.

In some embodiments, referring to FIGS. 6 and 7, the abutting portion 124 may be a pressing surface which is planar as a whole, or pressing surface of ridged shape protruding toward the sealing member 110. As compared with the planar surface, the ridged surface has a smaller contact area with the sealing member 110, and achieves a better compression effect on the sealing member 110, thereby enhancing the sealing effect.

In some embodiments, each linear motion member may be configured to comprise a screw body with external threads and coupled to the rotary motion assembly as a part of the linear motion member. The rotary motion assembly may be configured to comprise a primary gear, and at least one secondary gear in a number corresponding to that of the at least one linear motion member, the primary gear is meshed with the at least one secondary gear, and each secondary gear may be provided with a threaded hole matching the screw body.

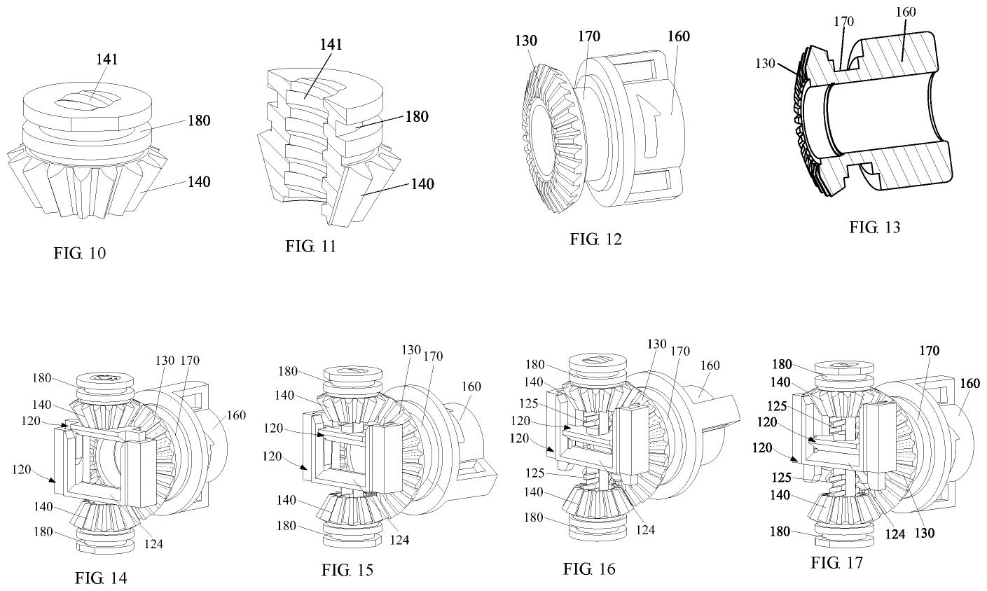

As exemplarily illustrated in FIGS. 2 to 17, the pressing member 120 may be configured to comprise a screw body 125 fixed to a side of the pressing portion 121 facing away from the abutting portion 124. The screw body 125, as a part of the pressing member 120, may be configured to be threadedly connected to a threaded hole 141 of the secondary gear 140. The threaded hole 141 may be axially disposed along a center of the secondary gear 140, and axial directions of the screw body 125 and the threaded hole 141 may be aligned with a radial direction of the sealing cavity 111. As exemplarily illustrated in FIGS. 2 and 3, an axial direction of the secondary gear 140 is the Y-axis direction, and of course may also be the Z-axis direction. The screw body 125 of each pressing member 120 is threadedly connected to one secondary gear 140. The axial position of the secondary gear 140 is fixed, that is, the secondary gear 140 cannot move along the transverse direction of the sealing cavity 111, and the primary gear 130 is disposed coaxially with the sealing cavity 111 and meshes with each secondary gear 140. By driving the primary gear 130 to rotate, each secondary gear 140 is rotated to drive the pressing member 120 for a linear motion in the transverse direction of the sealing cavity 111, so that the pressing portion 121 of the pressing member 120 extrudes or loosens the sealing member 110.

In this embodiment, when there are two linear motion members, two secondary gears 140 are available and located on the two opposite sides of the sealing member 110, respectively, and are threadedly connected to the screw bodies 125 of the two pressing members 120, respectively. When the primary gear 130 is driven to rotate, the two secondary gears 140 cause the two pressing portions 121 to move toward or away from each other via the screw bodies 125, so as to clamp or loosen the sealing member 110 quickly.

In this embodiment, a bevel gear set composed of the primary gear 130 and the secondary gear 140 is adopted to drive each pressing member 120 for simultaneous linear motions in the transverse direction of the sealing cavity 111, and a driving force is transmitted to the sealing member 110 by gear teeth and threads, thereby providing a stable, reliable and continuously adjustable transverse clamping force to the sealing member 110, and effectively preventing the pressing member 120 from moving undesirably in the Y-axis direction when not being operated. The hemostasis valve of this embodiment is simple in operation (it can be operated by a single operator), and the adjustment is convenient. However, the present disclosure is not limited thereto. In other embodiments, the linear motion of the pressing member 120 may also be driven by any other existing linear driving device.

In this embodiment, the pressing portion 121 and the screw body 125, as two parts of the linear motion member, may be an integral structure, or split structures connected to each other. In order to improve the structural strength and facilitate the assembly, the integral structure is preferred.

Further, the primary gear 130 and each secondary gear 140 may be both configured as bevel gears, which is convenient for spatial arrangement and the structure is more compact.

Further, as exemplarily illustrated in FIGS. 2 and 12, the primary gear 130 may be connected to a manual driving knob 160 and driven to rotate by the manual driving knob 160. For the convenience of the operator's operation, the manual driving knob 160 may be provided with an indication mark such as a rotation arrow. In order to be conveniently held by the operator, the manual driving knob 160 may be set as a flat structure or a ring-shaped structure, and an anti-slip structure such as anti-slip stripes or an anti-slip rubber sleeve may be provided on an outer side of the manual driving knob 160. However, the present disclosure is not limited thereto. In other embodiments, the manual driving knob 160 may also be replaced by an electric driving member such as an electric motor, which drives the primary gear 130 to rotate.

Further, as exemplarily illustrated in FIGS. 14 to 17, there are two pressing members 120, which are driven to move toward or away from each other by rotating the manual driving knob 160. When the manual driving knob 160 rotates for a certain angle, such as 180° or 360°, the pressing portions 121 of the two pressing members 120 fully approach each other.

As compared with a rotation of 360°, a rotation of 180° of the manual driving knob 160, which makes the pressing portions 121 of the two pressing members 120 fully approach each other, enables the operator to close the sealing cavity 111 in a shorter time, seal the medical device more efficiently, and similarly open the sealing cavity 111 more efficiently. For example, at the beginning, the pressing portions 121 of the two pressing members 120 fully approach each other. When the manual driving knob 160 is rotated for 180° in a direction opposite to that indicated by the indication mark of the arrow, the two pressing portions 121 are farthest apart. And then, when the manual driving knob 160 is rotated for 180° in the direction indicated by the indication mark of the arrow, the two pressing portions 121 fully approach each other.

FIG. 14 is a schematic diagram illustrating that the pressing portions 121 of the two pressing members 120 are furthest apart before the driving knob 160 being rotated. FIG. 15 is a schematic diagram illustrating that the two pressing members 120 approach each other when the driving knob 160 is rotated for about 60° in the direction indicated by the indication mark of the arrow. FIG. 16 is a schematic diagram illustrating that the two pressing members 120 further approach each other when the driving knob 160 is rotated for about 120° in the direction indicated by the indication mark of the arrow. FIG. 17 is a schematic diagram illustrating that the two pressing members 120 fully approach each other when the driving knob 160 is rotated for 180° in the direction indicated by the indication mark of the arrow.

Further, as exemplarily illustrated in FIGS. 4 and 12, an end of the primary gear 130 may be provided with a first connecting portion 170, through which the primary gear 130 is connected to the fixed case mounted with the hemostasis valve 10, e.g., the primary gear 130 is connected to the first housing 310 of the introducer 30. As exemplarily illustrated in FIGS. 1, 4, 10 and 11, an end of the secondary gear 140 may be provided with a second connecting portion 180, through which the secondary gear 140 is connected to the fixed case mounted with the hemostasis valve 10.

Specifically, both the first connecting portion 170 and the second connecting portion 180 may be annular grooves. Referring to FIG. 21, the fixed case may be provided with an annular supporting seat 314 matching the annular grooves. In order to support the smooth rotation of the primary gear 130 and the secondary gear 140, bearings may be further provided between the first connecting portion 170 and the fixed case, and between the second connecting portion 180 and the fixed case.

Preferably, the primary gear 130, the first connecting portion 170 and the manual driving knob 160 may be integrally formed, and the first connecting portion 170 may be located between the primary gear 130 and the manual driving knob 160. For the convenience of operation, the manual driving knob 160 may be provided outside the fixed case.

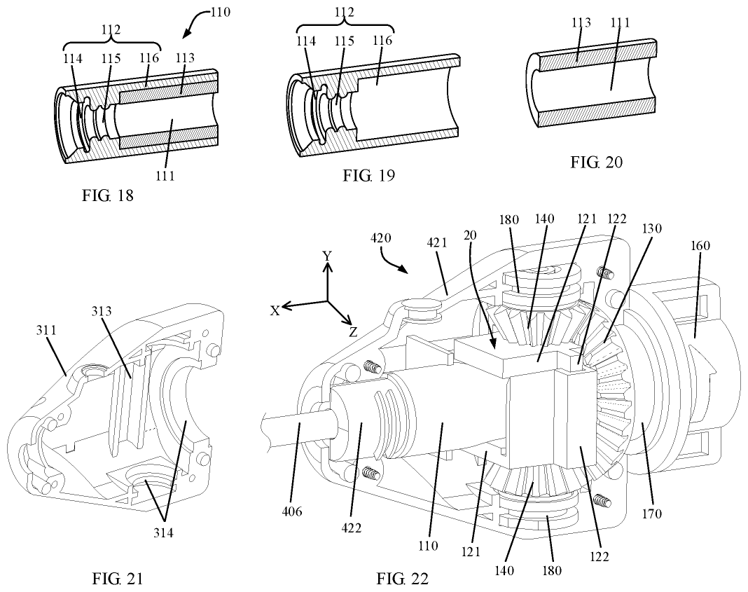

Preferably, the secondary gear 140 and the second connecting portion 180 may be integrally formed, and the threaded hole 141 inside the secondary gear 140 penetrates through the secondary gear 140 and the second connecting portion 180 along the axial direction. In some embodiments, referring to FIGS. 18 to 20, the sealing member 110 may comprise an outer sealing sleeve 112 and an inner sealing layer 113 fixed therein. A material hardness of the inner sealing layer 113 may be lower than that of the outer sealing sleeve 112, and the sealing cavity 111 may be formed inside the inner sealing layer 113.

In this embodiment, the material hardness of the outer sealing sleeve 112 is higher than that of the inner sealing layer 113, which not only ensures that the outer sealing sleeve 112 maintains a reliable connection with a tube such as a sheath tube (e.g., a first sheath tube 320) or a hub connecting tube 330, but also provide a reliable support for the inner sealing layer 113, while increasing a pressure applied to the medical device. Since being made of a soft material, the inner sealing layer 113 can effectively fill a gap between the medical device (e.g., a catheter and three positioning guide wires) inserted into the sheath tube and the sealing cavity 111, that is, it is convenient to seal around the inserted medical device. Therefore, the sealing member 110 with a double-layer structure of this embodiment can improve the sealing effect of sealing a plurality of independent medical devices within the sealing cavity 111.

In this embodiment, further, as exemplarily illustrated in FIGS. 18 and 19, the outer sealing sleeve 112 may comprise a tube connecting portion 114, a first sealing portion 115 and a mounting portion 116 arranged sequentially in an axial direction. The tube connecting portion 114 may be configured to connect a tube such as the first sheath tube 320 or the hub connecting tube 330. An inner wall of the first sealing portion 115 protrudes inward to form one or more annular sealing rings. The first sealing portion 115 forms a passive seal that is nonadjustable or only adjustable limitedly for the medical device through the sealing ring, and it is suitable for sealing a single medical device. The inner sealing layer 113 is fixed on an inner side of the mounting portion 116, and cooperates with the mounting portion 116 to form a second sealing portion of the sealing member 110. The pressing member 120 is disposed corresponding to an outer side of the mounting portion 116, and compresses the second sealing portion to transversely contract the sealing cavity 111. By clamping or loosening the second sealing portion, the pressing member 120 can seal or unseal a plurality of independent medical devices. Therefore, the second sealing portion can form a flexible and adjustable active seal for the medical devices.

In the present disclosure, the sealing member 110 may be constructed, in whole or in part, utilizing a variety of materials, such as, synthetic materials, natural materials and combinations thereof. In an embodiment, the sealing member 110 may be constructed of an elastic polymer such as silicone, polyurethane, latex or the like, or other suitable tube materials, including expanded polytetrafluoroethylene (ePTFE), silk, polyester weaves or other medical grade materials. By filling the pores of the tube material with an elastomer or other filling agents, the porous materials can be rendered less pervious to fluids and/or be made more lubricious.

When the sealing member 110 is constructed as a double-layer structure comprising the outer sealing sleeve 112 and the inner sealing layer 113, the materials of the outer sealing sleeve 112 and the inner sealing layer 113 may be selected from the materials of the sealing member 110, as long as the material hardness of the inner sealing layer 113 is lower than that of the outer sealing sleeve 112. The difference in hardness can be achieved by selecting materials with different hardness, or by selecting the same material but manufacturing with different hardness.

For example, the outer sealing sleeve 112 may be made of silicone (the hardness is A40-60), and the inner sealing layer 113 may be made of silicone (the hardness is A0-30).

Referring to FIGS. 22 and 23, it is another aspect of the present disclosure to provide a hemostasis valve 20, which is different from the hemostasis valve 10 in that the pressing portion 121 of at least one linear motion member of the hemostasis valve 20 is further provided with a clamping portion that protrudes toward a central axis of the sealing member 110. As exemplarily illustrated in FIGS. 22 and 23, the clamping portion may be a clamping strip 210. In the transverse direction of the sealing cavity 111, the clamping portion and the sealing member 110 do not oppose each other. That is, the pressing portion 121 of the hemostasis valve 20 is provided with an abutting portion 124 and a clamping portion, wherein the abutting portion 124 oppose the sealing member 110 and is configured to change the dimension of the sealing cavity 111, while the clamping portion does not oppose the sealing member 110 and is configured to clamp and fix the medical device passing through the sealing cavity 111 outside the sealing member 110. Under the driving of the rotary motion assembly, when the abutting portion 124 compresses the sealing member 110, the clamping portion clamps and fixes the medical device, so that the hemostasis valve 20 has not only a sealing function, but also a function of assisting in removing the medical device.

For example, when the medical device passes through the hemostasis valve 20 of this embodiment, the medical device may be pulled out of a patient's body by directly drawing the hemostasis valve 20, without adding any additional device for clamping and fixing the medical device, which facilitates the operation, saves the time and improves the operation efficiency.

The hemostasis valve 20 of this embodiment is particularly suitable for sealing medical devices made of metal and assisting in removing the same. Although the hemostasis valve 10 can seal the medical device made of metal, a friction force between the sealing member 110 made of elastic polymer and the medical device made of metal is small when the medical device is pulled out of the patient, and it is difficult to transmit the pulling force to the medical device made of metal, which is not conducive to pulling out the medical device. However, the clamping portion in this embodiment can clamp the medical device and increase the friction force between the pressing portion 121 of the pressing member 120 and the medical device. Therefore, the hemostasis valve 20 of this embodiment is suitable for hemostasis sealing and assistance in removing the medical device made of metal (e.g., one or more positioning guide wires) or nonmetal (e.g., one or more catheters).

As exemplarily illustrated in FIGS. 22 and 23, the clamping portion may be a long clamping strip 210, which is convenient for clamping the medical device. However, the present disclosure is not limited thereto. In other embodiments, any other clamping portion such as a clamping block may be adopted.

In some embodiments, the hemostasis valve 20 may comprise two linear motion members. The pressing portions 121 of the two linear motion members (i.e., the pressing members 120) jointly extrude the sealing member 110 from the two opposite sides of the sealing member 110.

The pressing portion 121 of each pressing member 120 may be provided with one or more clamping strips 210 arranged at intervals along the axial direction of the sealing cavity 111. The extending direction of each clamping strip 210 may not be parallel to the longitudinal direction of the sealing cavity 111, that is, an included angle between each clamping strip 210 and the longitudinal direction of the sealing cavity 111 may be greater than 0° and less than 180°. The clamping strips 210 on the two pressing members 120 can jointly clamp and fix the medical devices.

However, the present disclosure is not limited thereto, and the number of the linear motion members in this embodiment may be one or more than two. For example, when there is one linear motion member, a clamping strip fitted with the clamping strip 210 of the linear motion member may be provided on the fixed case (e.g., a second housing 421 in FIG. 22) where the hemostasis valve 20 is mounted. When the pressing member 120 of the hemostasis valve 20 compresses the sealing member 110, the clamping strip 210 of the pressing member 120 and the clamping strip on the fixed case jointly clamp and fix the medical devices.

Further, referring to FIG. 24, in the longitudinal direction of the sealing cavity 111, the clamping strips 210 of the two pressing members 120 may be arranged alternatively, so as to further improve the clamping stability of the clamping strip 210 to the medical devices.

Further, the extending direction of each clamping strip 210 may be perpendicular to the longitudinal direction of the sealing cavity 111. As exemplarily illustrated in FIG. 23, a length direction of the clamping strip 210 is the Z-axis direction, so as to further improve the clamping stability of the clamping strip 210 to the medical devices.

As exemplarily illustrated in FIGS. 23 and 24, the surface of the pressing portion 121 facing the sealing member 110 may comprise two areas adjacent to each other in the X-axis direction, as illustrated in FIGS. 25 to 28, wherein one area serves as the abutting portion 124, and the other area is provided with the clamping strip 210. In this embodiment, a longitudinal width of the pressing portion 121 in the X-axis direction may be greater than that of the pressing portion 121 not provided with the clamping strip 210 of the hemostasis valve 10, so as to facilitate the arrangement of a plurality of clamping strips 210.

Further, the clamping strip 210 may be integrally formed with the pressing member 120, that is, the clamping strip 210 may be made of the same material as the pressing member 120. Preferably, the clamping strip 210 is made of metal. However, the present disclosure is not limited thereto. The clamping strip 210 and the pressing member 120 may also be split structures and fixedly connected to each other. The materials of the clamping strip 210 and the pressing member 120 may be the same or different. For example, the clamping strip 210 is made of metal and the pressing member 120 is made of polymer.

Further, a clamping surface of the clamping strip 210 in contact with the medical devices may be further provided with an anti-slip structure, so as to increase the friction force between the clamping strip 210 and the medical devices through the anti-slip structure. For example, the anti-slip structure may be a concave-convex structure or any other existing structure which can increase the friction force.

Other structures, working principles and advantageous effects of the hemostasis valve 20 are the same as those of the hemostasis valve 10, and will not be described in detail.

The hemostasis valve 10 and the hemostasis valve 20 may be adopted according to actual needs, that is, any of the aforementioned hemostasis valves may be employed in the surgical operation procedure, and of course, both hemostasis valves may also be employed in cooperation at the same time.

Referring to FIGS. 1 to 5, it is another aspect of the present disclosure to provide an introducer 30, which may comprise a hemostasis valve. The hemostasis valve may be the hemostasis valve 10.

In some embodiments, referring to FIGS. 2 to 4, the introducer 30 may further comprise a housing (referred to as a first housing 310) and a sheath tube (referred to as a first sheath tube 320). The sealing member 110 and the linear motion member of the hemostasis valve 10 may be disposed inside the first housing 310. The first sheath tube 320 may be connected to the sealing member 110. The first sheath tube 320 may be arranged coaxially with and communicated with the sealing cavity 111.

Further, referring to FIGS. 2 to 4, the primary gear 130 and the secondary gear 140 of the hemostasis valve 10 may be disposed inside the first housing 310. The manual driving knob 160 may be provided outside the first housing 310. The primary gear 130 and the secondary gear 140 may be rotatably connected to the first housing 310. The first housing 310 not only supports the primary gear 130 and the secondary gear 140, but also fixes the axial positions thereof to prevent the primary gear 130 and the secondary gear 140 from moving in their respective axial directions.

Specifically, referring to FIGS. 3 and 21, the primary gear 130 may be connected to the first housing 310 through the first connecting portion 170, and the secondary gear 140 may be connected to the first housing 310 through the second connecting portion 180. The first housing 310 may be provided with an annular supporting seat 314 matching the first connecting portion 170 and the second connecting portion 180.

Further, referring to FIGS. 3 and 21, the first housing 310 may be composed of a first case 311 and a second case 312 connected to each other. When the first case 311 and the second case 312 are connected to each other, a space is formed there-between for mounting the sealing member 110, the pressing member 120, the primary gear 130 and the secondary gear 140. For the convenience of assembly, the first case 311 and the second case 312 may be detachably and fixedly connected to each other, for example, by screws. In order to guide the first linear motion member to move smoothly, a guide groove 313 for accommodating the first guide rod 122 of the first linear motion member may be provided on the inner wall of the first housing 310. When the first linear motion member moves in the transverse direction of the sealing cavity 111, the first guide rod 122 of the first linear motion member smoothly slides along the guide groove 313.

In this embodiment, the material of the first housing 310 may be polymethyl methacrylate (PMMA), polystyrene (PS), acrylonitrile butadiene styrene (ABS), polyvinyl chloride (PVC), modified polyethylene terephthalate glycerol (PETG), cellulose acetate butyrate (CAB), polyethylene (PE), high density polyethylene (HDPE), low density polyethylene (LDPE or LLDPE), polypropylene (PP), polycarbonate (PC), modified polyphenylene oxide (MPPO), polyphenylene ether (PPE), thermoplastic polyurethane (TPU), polyamide (PA or nylon), polyoxymethylene (POM), polyethylene terephthalate (PET, thermoplastic polyester), polybutylene terephthalate (PBT, thermoplastic polyester), ultra-high molecular weight polyethylene (UHMWPE), fluorinated ethylene-propylene (FEP) or any other medical grade polymer commonly known in the art. The material of the first housing 310 is also suitable for the primary gear 130, the secondary gear 140 and the pressing assembly.

In some embodiments, referring to FIGS. 2 to 4, the introducer 30 may further comprise a hub connecting tube 330 and a side tube (referred to as a first side tube 340), and the first sheath tube 320 and the sealing member 110 may be connected to each other through the hub connecting tube 330. The first side tube 340 may be disposed to be perpendicular to the longitudinal direction of the sealing cavity 111, and may be connected to the hub connecting tube 330. The first side tube 340 may serve as a flushing port. Of course, the introducer 30 may also comprise other commonly known fittings such as a flushing valve seat.

Specifically, referring to FIGS. 2, 3, 4 and 18, the hub connecting tube 330 may comprise a main tube 331 and a branch tube 332 which are communicated with each other. One end of the main tube 331 may be connected to the first sheath tube 320, the other end of the main tube 331 may be connected to the tube connecting portion 114 of the outer sealing sleeve 112 of the sealing member 110, and the main tube 331, the first sheath tube 320 and the sealing member 110 may be coaxially arranged. The branch tube 332 may be perpendicular to the main tube 331, with one end connected to an outer side wall of the main tube 331 and the other end connected to the first side tube 340. The hinge connecting tube 330 may be located inside the first housing 310, and the first sheath tube 320 and the first side tube 340 may be located outside the first housing 310.

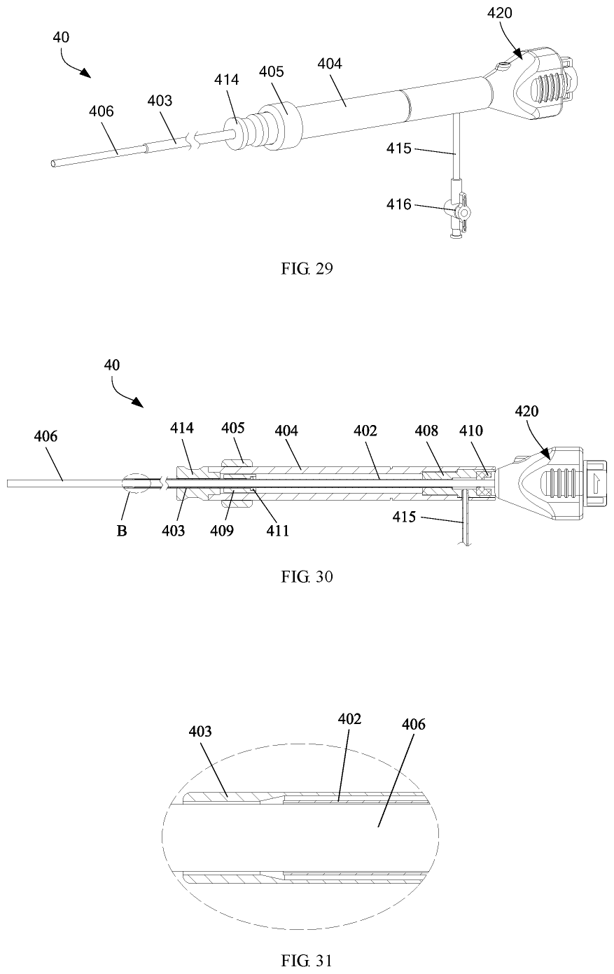

With reference to FIGS. 29 to 33, it is another aspect of the present disclosure to provide a retrieval device 40 for retrieving an implant in a human body, for example, retrieving a cardiovascular implant such as an aortic valve that has been implanted in a patient's body while being unsuitable and needed to be replaced. The retrieval device 40 may comprise an elastic retrieval net 401, an inner tube 402, an outer tube 403, a handle 404, a slider 405, a sheath tube (referred to as a second sheath tube 406), and a sealing assembly 420 with a hemostasis valve which is the hemostasis valve 10 (refer to FIGS. 2 and 4) or the hemostasis valve 20 (refer to FIGS. 22 and 23). The retrieval net 401 has an opened state in which the retrieval net 401 is funnel-shaped or umbrella-shaped and can capture an implant, and a closed state in which the retrieval net 401 can tightly wrap the implant inside, which is convenient for an operator to pull the implant out of the human body. The inner tube 402 is connected to the retrieval net 401. Referring to FIGS. 34 and 35, the outer tube 403 sleeves the inner tube 402, while the outer tube 403 and the inner tube 402 are in sealing fit and movable axially relative to each other. The retrieval net 401 can be moved into or out of the outer tube 403 by driving the outer tube 403 and the inner tube 402 to move axially relative to each other.

When located inside the outer tube 403, the retrieval net 401 is in the closed state under the constraint of the outer tube 403. When located outside the outer tube 403, the retrieval net 401 naturally expands into the opened state. The handle 404 may be cylindrical and sleeves the outer tube 403. The slider 405 may be disposed outside the handle 404 and is in sliding fit therewith. The slider 405 may be configured to be slidable along the axial direction of the inner tube 402. One of the outer tube 403 and the inner tube 402 is fixedly connected to the handle 404 and the other is fixedly connected to the slider 405. The outer tube 403 and the inner tube 402 are driven to move axially relative to each other by operating the slider 405. The second sheath tube 406 may be disposed to run through the inner tube 402 and in sealing fit therewith. The sealing member 110 of the hemostasis valve may be connected to the second sheath tube 406, and the sealing cavity 111 of the sealing member 110 may be coaxially arranged and communicated with the second sheath 406.

The medical device in the second sheath tube 406 (e.g., the positioning guide wire 60 illustrated in FIG. 23) passes through the sealing cavity 111 of the sealing assembly 420, and the implant in the human body is connected to the medical devices in the second sheath tube 406, wherein the hemostasis valve can prevent a blood loss during implant retrieval.

An operation method of retrieving an implant is as follows:
firstly, sliding the slider 405 to move the retrieval net 401 to the outside of the outer tube 403, at which time the retrieval net 401 is in the opened state;
then, pulling the hemostasis valve to cause the medical devices and the implant to move toward the stretched retrieval net 401 until the implant moves into the retrieval net 401;
next, pulling the handle entirety to move toward a proximal end (the handle entirety comprises the handle 404, the slider 405 and the hemostasis valve, and the slider 405 can be prevented from moving relative to the handle 404 by hand pressing or in other ways), so that the retrieval net 401 and the implant move into the sheath tube of the introducer 30 (e.g., the first sheath tube 320 of the introducer 30);
continuing pulling the handle entirety to move toward the proximal end until the entirety of the retrieval device 40 and the implant are pulled out of the human body.

For example, the retrieval net 401 may be a net structure formed by weaving elastic materials, or by engraving a whole tube by laser engraving, etc. The retrieval net 401 may be made of stainless steel, nitinol, or any other suitable biomedical material, preferably nitinol. Specifically, the retrieval net 401 may be coated with a hydrophilic and/or resistance-reducing coating, such as PTFE coating. The retrieval net 401 may be preformed to form a memory structure, which is funnel-shaped in an unconstrained state, while deformed randomly in a constrained state, e.g., shrinking into a cylinder-like shape in the outer tube 403. The rear end of the retrieval net 401 and the front end of the inner tube 402 are fixed together by bonding, melting, sewing, or other conventional means.

In a preferred embodiment, referring to FIGS. 22 to 28, the hemostasis valve of the sealing assembly 420 may be the hemostasis valve 20. Since the medical devices connected to the implant are usually positioning guide wires made of metal, the friction force between the positioning guide wires and the clamping strip 210 can be increased by adopting the hemostasis valve 20 with the clamping strip 210, which is beneficial to the withdrawing of the positioning guide wires when the sealing assembly 420 is pulled. As exemplarily illustrated in FIGS. 29 and 30, the sealing assembly 420 may be provided at an end of the handle 404 distal to the retrieval net 401.

Further, referring to FIGS. 22 and 23, the sealing assembly 420 may further comprise a housing (referred to as a second housing 421) in which the sealing member 110, the linear motion member, the primary gear 130 and the secondary gear 140 of the hemostasis valve 20 are disposed. The manual driving knob 160 of the hemostasis valve 20 may be disposed outside the second housing 421. In this embodiment, the structure and the material of the second housing 421 in addition to connection and fitting relationships with the hemostasis valve 20 can be set with reference to that of the first housing 310 of the introducer 30, and will not be described in detail.

Further, referring to FIGS. 22 and 23, the sealing assembly 420 may further comprise a connecting tube 422 disposed in the second housing 421, wherein one end of the connecting tube 422 may be connected to the second sheath tube 406, and the other end thereof may be connected to the tube connecting portion 114 of the outer sealing sleeve 112 of the hemostasis valve 20.

In some embodiments, referring to FIGS. 30 and 33, the retrieval device 40 may further comprise an inner tube seat 408 and an outer tube seat 409. The distal end of the inner tube 402 is connected to the retrieval net 401, and the proximal end of the inner tube 402 is connected to the inner tube seat 408. The proximal end of the outer tube 403 is connected to the outer tube seat 409, and the distal end of the outer tube 403 allows the retrieval net 401 to enter or move out. Both the inner tube seat 408 and the outer tube seat 409 are disposed inside the handle 404. One of the inner tube seat 408 and the outer tube seat 409 is fixedly connected to the handle 404, and the other is fixedly connected to the slider 405. A first hemostasis sealing member 410 for sealing between the inner tube 402 and the second sheath tube 406 may be disposed in the inner tube seat 408, and a second hemostasis sealing member 411 for sealing between the outer tube 403 and the inner tube 402 may be disposed in the outer tube seat 409. For example, the first hemostasis sealing member 410 and the second hemostasis sealing member 411 are sealing rings.

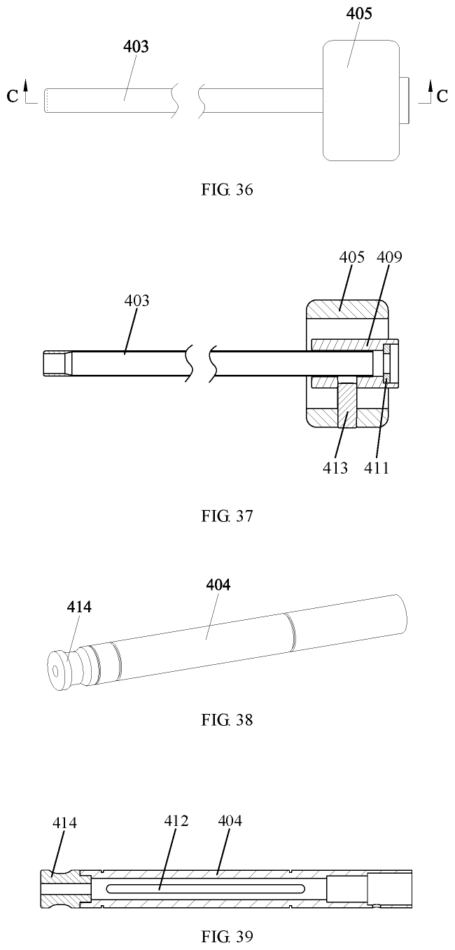

In some embodiments, referring to FIGS. 36 to 39, the handle 404 may be provided with a sliding groove 412, and the length direction of the sliding groove 412 is the longitudinal direction of the inner tube 402. The slider 405 is connected to a pin 413 which passes through the sliding groove 412 and extends into the handle 404. The slider 405 is connected to the inner tube 402 or the outer tube 403 through the pin 413. The inner tube 402 and the outer tube 403 are moved axially relative to each other by sliding the slider 405 along the sliding groove 412.

In some examples, the handle 404 may be fixedly connected to the inner tube seat 408, and the slider 405 may be fixedly connected to the outer tube seat 409 through the pin 413. When the slider 405 slides, the slider 405 and the pin 413 cause the outer tube seat 409 and the outer tube 403 to move axially together relative to the inner tube 402. For example, the slider 405 is an annular sliding block.

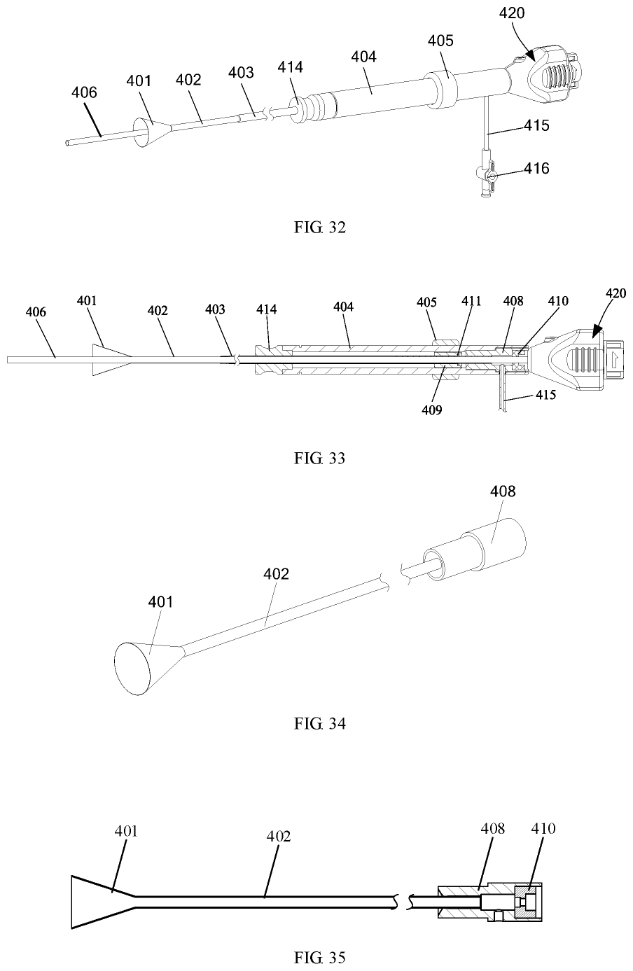

Further, referring to FIGS. 32, 38 and 39, in order to provide a support and guidance for the outer tube 403 and avoid instability of the outer tube 403 during movement, a guide sleeve 414 may be provided at the front end of the handle 404. The inner diameter of the guide sleeve 414 is slightly greater than the outer diameter of the outer tube 403, and the outer tube 403 is disposed to penetrate through the guide sleeve 414. The handle 404 and the guide sleeve 414 may be an integral structure or split structures.

Further, the retrieval device 40 may further comprise a second side tube 415 and a flush valve 416 provided thereon. The second side tube 415 may be disposed to be perpendicular to a longitudinal direction of the handle 404, connected to a side wall of the handle 404, and communicated with the inner tube seat 408.

In other examples, the handle 404 may be fixedly connected to the outer tube seat 409, and the slider 405 may be fixedly connected to the inner tube seat 408 through the pin 413. When the slider 405 slides, the slider 405 and the pin 413 cause the inner tube seat 408 and the inner tube 402 to move axially together relative to the outer tube 403.

The retrieval device 40 of the present disclosure is suitable for retrieving a cardiovascular implant, and particularly for retrieving a cardiovascular prosthetic valve with an inflatable cuff of US patent with a Publication No. US 2009/0088836 A1.

Those described above are just specific embodiments of the present disclosure, and are not intended to limit the scope of the present disclosure. Any equivalent change and modification made by those skilled in the art without departing from the concept and principle of the present disclosure should fall within the protection scope of the present disclosure. Furthermore, it should be noted that the constituent parts of the present disclosure are not limited to the above overall application, and the technical features described in the specification of the present disclosure can be selectively adopted alone or in combination according to actual needs. Therefore, the present disclosure naturally covers any other combination and specific application related to the inventive points of the present disclosure.

1. A hemostasis valve, comprising:
a sealing member having a longitudinal axis and defining a sealing cavity which is transversely contractible;
a pressing assembly disposed at an outer periphery of the sealing member and configured for linear motion in a transverse direction of the sealing cavity perpendicular to the longitudinal axis to contract a dimension of the sealing cavity; and
a rotary motion assembly coupled to the pressing assembly and configured to convert rotary motion into the linear motion perpendicular to the longitudinal axis of the pressing assembly through the coupling.

2. The hemostasis valve according to claim 1, wherein the pressing assembly is configured to comprise at least one linear motion member, a part of which is coupled to the rotary motion assembly, and another part of which provides an abutting portion configured to abut the sealing member to contract the dimension of the sealing cavity, and the rotary motion assembly is configured to convert the rotary motion into the linear motion of the at least one linear motion member through the coupling.

3. The hemostasis valve according to claim 2, wherein said another part of the at least one linear motion member is provided with a pressing portion which comprises the abutting portion.

4. The hemostasis valve according to claim 3, wherein,
the at least one linear motion member comprises a screw body with threads;
the rotary motion assembly comprises a primary gear and at least one secondary gear in a number corresponding to that of the at least one linear motion member, the primary gear being meshed with the at least one secondary gear;
the at least one secondary gear is provided with threads configured to engage with the threads of the screw body.

5. The hemostasis valve according to claim 4, wherein the primary gear and the at least one secondary gear are configured as bevel gears.

6. The hemostasis valve according to claim 3, wherein the abutting portion is a pressing surface which is planar as a whole, or pressing surface of ridged shape.

7. The hemostasis valve according to claim 3, wherein,
the at least one linear motion member is configured to comprise a first linear motion member and a second linear motion member which are oppositely disposed;
the first linear motion member is configured to comprise a first pressing portion having a first abutting portion;
the second linear motion member is configured to comprise a second pressing portion having a second abutting portion.

8. The hemostasis valve according to claim 7, wherein,
the first pressing portion is provided with a first guide rod, and the second pressing portion is provided with a second guide rod;
the first guide rod and the second guide rod extend and guide in the transverse direction of the sealing cavity;
the first guide rod is in sliding fit with the second guide rod to guide the linear motion.

9. The hemostasis valve according to claim 7, wherein at least one of the first pressing portion and the second pressing portion is further provided with a clamping portion protruding toward the central axis of the sealing member, and the clamping portion and the sealing member do not oppose each other in the transverse direction of the sealing cavity.

10. The hemostasis valve according to claim 9, wherein the first pressing portion and the second pressing portion are both provided with the clamping portions arranged at intervals along a longitudinal direction of the sealing cavity;
the extending direction of the clamping portion is not parallel to the longitudinal direction of the sealing cavity; and/or
the clamping portion of the first pressing portion and the clamping portion of the second pressing portion are arranged alternatively.

11. The hemostasis valve according to claim 10, wherein the extending direction of the clamping portion is perpendicular to the longitudinal direction of the sealing cavity.

12. The hemostasis valve according to claim 1, wherein,
the sealing member comprises an outer sealing sleeve and an inner sealing layer fixed to the inside of the outer sealing sleeve, the material hardness of the inner sealing layer being lower than that of the outer sealing sleeve, and the sealing cavity being formed inside the inner sealing layer;
the outer sealing sleeve comprises a tube connecting portion, a first sealing portion and a mounting portion arranged sequentially in an axial direction of the outer sealing sleeve, and the inner wall of the first sealing portion protrudes inward to form one or more sealing rings;
the inner sealing layer is fixed to the inner side of the mounting portion and forms a second sealing portion of the sealing member together with the mounting portion, and the pressing assembly is configured to change the dimension of the sealing cavity corresponding to the second sealing portion through the linear motion.

13. An introducer comprising a housing and a sheath tube, and further comprising the hemostasis valve according to claim 1 and a driving knob for operating the hemostasis valve;
the hemostasis valve is disposed inside the housing, the sheath tube is connected to the sealing member, and the sheath tube is arranged coaxially with and communicated with the sealing cavity;
the driving knob is disposed outside the housing and attached to the rotary motion assembly.

14. A retrieval device for retrieving a cardiovascular implant, comprising:
a retrieval net having an opened state and a closed state;
an inner tube connected to the retrieval net;
an outer tube sleeving the inner tube, with the outer tube and the inner tube being in sealing fit and movable axially relative to each other, and the retrieval net being moved into or out of the outer tube by driving the outer tube and the inner tube to move axially relative to each other;
a handle sleeving the outer tube;
a slider disposed outside the handle and in sliding fit with the handle, one of the outer tube and the inner tube being fixedly connected to the handle and the other being fixedly connected to the slider, and the outer tube and the inner tube being driven to move axially relative to each other by sliding the slider;
a sheath tube running through the inner tube and in sealing fit with the inner tube; and
the hemostasis valve according to claim 1, with the sealing member being connected to the sheath tube, and the sealing cavity being communicated with the sheath tube.

15. The retrieval device according to claim 14, wherein,
the retrieval device further comprises an inner tube seat and an outer tube seat which are both disposed inside the handle;
an end of the outer tube distal to the retrieval net is connected to the outer tube seat, and two ends of the inner tube are connected to the retrieval net and the inner tube seat, respectively;
one of the inner tube seat and the outer tube seat is fixedly connected to the handle and the other is fixedly connected to the slider;
a first hemostasis seal for sealing between the inner tube and the sheath tube is disposed in the inner tube seat, and a second hemostasis seal for sealing between the outer tube and the inner tube is provided in the outer tube seat.

16. The retrieval device according to claim 14, wherein the handle is provided with a sliding groove which extends in a longitudinal direction of the inner tube, the slider is connected to a pin which runs through the sliding groove and extends into the handle, and the slider is connected to the inner tube or the outer tube through the pin.

17. The hemostasis valve according to claim 1, wherein when the pressing assembly moves linearly in the transverse direction of the sealing cavity perpendicular to the longitudinal axis to contract a dimension of the sealing cavity, the pressing assembly changes the shape of the sealing cavity.

18. The hemostasis valve according to claim 17, wherein the pressing assembly is configured to change the shape of the sealing cavity from a circular shape to a non-circular shape.
